(19) United States
(12) Patent Application Publication
(10) Pub. No.: US 2023/0414367 A1
(43) Pub. Date: Dec. 28, 2023

(54) METHOD OF DESIGNING PATIENT-SPECIFIC CRANIOPLASTY IMPLANTS

(71) Applicant: University of Cincinnati, Cincinnati, OH (US)

(72) Inventors: Vysakh Venugopal, Cincinnati, OH (US); Matthew McConaha, CIncinnati, OH (US); Omkar Ghalsasi, Melbourne, FL (US); Alice Xu, Novi, MI (US); Sam Anand, West Chester, OH (US); Jonathan Forbes, Cincinnati, OH (US); Joshua Cheng, Cincinnati, OH (US); Manish Raj Aryal, Cincinnati, OH (US)

(73) Assignee: University of Cincinnati, Cincinnati, OH (US)

(21) Appl. No.: 18/212,873

(22) Filed: Jun. 22, 2023

Related U.S. Application Data
(60) Provisional application No. 63/354,638, filed on Jun. 22, 2022.

(51) Int. Cl. A61F 2/28; G06F 30/20

(52) U.S. Cl. CPC A61F 2/2875; G06F 30/20; A61F 2002/30952

(57) ABSTRACT

Methods, processing systems, and computer-readable mediums for automated designing of patient-specific cranial implants may include extracting pixel data from a DICOM file; generating a virtual skull model based on the pixel data; identifying a mid-sagittal plane of the virtual skull model; identifying a surgical hole in the virtual skull model; mirroring a reference side of the virtual skull model onto a surgical hole side of the virtual skull model; subtracting the surgical hole side from the mirrored reference side to generate a virtual cranial implant; and generating a virtual two-part mold based on the virtual cranial implant. A physical two-part mold can be 3D printed based on the virtual two-part mold, and a physical cranial implant can be constructed using the physical two-part mold.

### CROSS REFERENCE TO RELATED APPLICATIONS

The present application claims the benefit of U.S. Provisional Application Ser. No. 63/354,638, filed Jun. 22, 2022, which is hereby incorporated by reference in its entirety.

### TECHNICAL FIELD

Aspects of the disclosure provide methods, processing systems, and computer-readable mediums for automated designing of patient-specific cranial implants.

### BACKGROUND

Following a traumatic injury to the head, intracranial pressure can increase as injured brain tissue swells inside the fixed intracranial compartment. Increasing levels of intracranial pressure can result in detrimental secondary injury to the brain. If intracranial pressure continues to rise beyond dangerous levels, brain tissue can herniate into another compartment, resulting in a life-threatening herniation syndrome. To prevent/relieve the buildup of dangerous levels of intracranial pressure, a procedure called a decompressive craniectomy is performed. In this surgery, a large portion of the skull is surgically removed, providing space for the brain to swell outwards through the created defect. Once the swelling has alleviated and the patient has recovered from the trauma and surgery, the cranial defect should be repaired using a procedure known as a cranioplasty.

While the use of the patient's bone flap was previously a standard practice, artificial implants have now been found to be superior in long-term outcomes with fewer complications such as resorption and infection. The difference in the cost is minimal when factoring in operating room time, the hardware used, length of hospital stay, and management of medical complications. The ideal implant is reasonably priced and cosmetically precise. In developed countries, third-party vendors can provide customized implants by using a thin-cut, post-operative computed tomography scan of the patient's skull. However, this process is costly and most hospitals in developing countries do not have the financial resources to utilize this option. Instead, neurosurgeons in developing countries routinely manually mold an implant out of polymethyl methacrylate bone cement or titanium mesh in the operating room.

Accordingly, a need exists for alternative methods for designing cost-effective and cosmetically precise patient specific cranial implants.

### SUMMARY

Additional features and advantages of the present disclosure will be set forth in the detailed description, which follows, and in part will be apparent to those skilled in the art from that description or recognized by practicing the embodiments described herein, including the detailed description, which follows the claims, as well as the appended drawings.

In one embodiment, a method includes imaging a head of a patient to generate a DICOM file including pixel data of the imaged head; extracting a virtual skull model from the pixel data of the DICOM file, the virtual skull model having a reference side and a surgical hole side; determining a location of a mid-sagittal plane of the virtual skull model; identifying a location of the zygomatic bone in the virtual skull model and defining an inferior boundary of the virtual cranial implant based on the location of the zygomatic bone; determining a distance to an inside surface and an outside surface of the virtual skull model on both the reference side and surgical hole side; identifying a surgical hole and the surgical hole side of the virtual skull model based on the distance to the inside surface and the distance to the outside surface of the virtual skull model on both the reference side and surgical hole side of the virtual skull model; identifying a ridge of the surgical hole on the surgical hole side of the virtual skull model; aligning the inside surface and the outside surface of both the reference side and the surgical hole side of the virtual skull model to generate an aligned inner surface and an aligned outer surface on the reference side of the virtual skull model; generating a virtual cranial implant based on the surgical hole, the aligned inner surface on the reference side of the virtual skull model, and the aligned outer surface on the reference side of the virtual skull model; and generating a virtual two-part mold based on the virtual cranial implant.

In another embodiment, a non-transitory computer readable medium includes instructions that, when executed by a processor, cause the processor to perform operations comprising: extracting pixel data from a DICOM file; generating a virtual skull model based on the pixel data; identifying a mid-sagittal plane of the virtual skull model; identifying an inferior boundary of the virtual skull model; identifying a surgical hole in the virtual skull model; mirroring a reference side of the virtual skull model onto a surgical hole side of the virtual skull model; subtracting the surgical hole side from the mirrored reference side to generate a virtual cranial implant; and generating a virtual two-part mold based on the virtual cranial implant.

In yet another embodiment, a method includes: receiving a DICOM file containing image data representative of an image of a patient's head through a graphical user interface coupled to a processor and displayed on a display; generating a virtual skull model based on the image data with the processor; identifying a mid-sagittal plane of the virtual skull model with the processor; identifying a surgical hole in the virtual skull model with the processor; mirroring a reference side of the virtual skull model onto a surgical hole side of the virtual skull model with the processor; subtracting the surgical hole side from the mirrored reference side to generate a virtual cranial implant with the processor; scaling the virtual cranial implant by a user defined scale factor through the graphical user interface; generating a virtual two-part mold based on the scaled virtual cranial implant with the processor; generating a pin-lock mechanism on the virtual two-part mold with the processor, the pin-lock mechanism comprising a plurality of pin-receiving members disposed on one half of the two-part mold and a plurality of pins disposed on the other half of the two-part mold; specifying a length of the plurality of pins of the pin-lock mechanism through the graphical user interface; generating an STL file of the virtual two-part mold and pin-lock mechanism with the processor; printing a physical two-part mold and pin-lock mechanism from the STL file using an additive manufacturing apparatus; and forming a physical cranial implant using the physical two-part mold and pin-lock mechanism.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein, and together with the description, explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a method for designing and constructing a cranial implant, according to one or more embodiments shown and described herein;

FIG. 2 schematically depicts an anatomical coordinate system and a Cartesian coordinate system, according to one or more embodiments shown and described herein;

FIG. 3 schematically depicts a matrix representation visualization of anatomical and Cartesian coordinates, according to one or more embodiments shown and described herein;

FIG. 4 schematically depicts an iterative process flow to identify a midsagittal plane based on a symmetry metric, according to one or more embodiments shown and described herein;

FIG. 5A schematically depicts an output plot of a plane position obtained using the midsagittal plane identification process of FIG. 4, according to one or more embodiments shown and described herein;

FIG. 5B schematically depicts an example identified midsagittal plane using the midsagittal plane identification process of FIG. 4, according to one or more embodiments shown and described herein;

FIG. 5C schematically depicts another example identified midsagittal plane using the midsagittal plane identification process of FIG. 4, according to one or more embodiments shown and described herein;

FIG. 6 schematically depicts a ray tracing approach used to identify the inner and outer surfaces of the skull on both sides of the midsagittal plane, according to one or more embodiments shown and described herein;

FIG. 7A schematically depicts a color map plot showing the distance of the inner surface of the reference/pristine side of the skull from the midsagittal plane, according to one or more embodiments shown and described herein;

FIG. 7B schematically depicts a color map plot showing the distance of the outer surface of the reference/pristine side of the skull from the midsagittal plane, according to one or more embodiments shown and described herein;

FIG. 7C schematically depicts a color map plot showing the distance of the inner surface of the hole side of the skull from the midsagittal plane, according to one or more embodiments shown and described herein;

FIG. 7D schematically depicts a color map plot showing the distance of the outer surface of the hole side of the skull from the midsagittal plane, according to one or more embodiments shown and described herein;

FIG. 8A schematically depicts binary projection of the pristine side of the skull on the midsagittal plane, according to one or more embodiments shown and described herein;

FIG. 8B schematically depicts binary projection of the hole side of the skull on the midsagittal plane, according to one or more embodiments shown and described herein;

FIG. 8C schematically depicts a connected-component labeling output of the pristine side of the skull on the midsagittal plane, according to one or more embodiments shown and described herein;

FIG. 8D schematically depicts a connected-component labeling output of the hole side of the skull on the midsagittal plane, according to one or more embodiments shown and described herein;

FIG. 9 schematically depicts a 4-pixel averaging kernel applied on a sample offset matrix, according to one or more embodiments shown and described herein;

FIG. 10A schematically depicts an output plot of a sample offset matrix, according to one or more embodiments shown and described herein;

FIG. 10B schematically depicts an smoothened output plot of the 4-pixel averaging kernel process of FIG. 4, according to one or more embodiments shown and described herein;

FIG. 11 schematically depicts a mirroring operation and a ridge that forms the thickness of the skull, according to one or more embodiments shown and described herein;

FIG. 12A schematically depicts the thickness of the skull at an identified ridge portion, according to one or more embodiments shown and described herein;

FIG. 12B schematically depicts the sudden decrease in distance of the outer surface of the skull from the midsagittal plane, according to one or more embodiments shown and described herein;

FIG. 13A schematically depicts the difference in the distance of outer surfaces with respect to both sides of the midsagittal plane, according to one or more embodiments shown and described herein;

FIG. 13B schematically depicts a tracking of the ridge based on the difference in the distances of the outer surfaces of the reference side of the skull and the hole side of the skull, according to one or more embodiments shown and described herein;

FIG. 14 schematically depicts the dilation of a sample matrix based on the Manhattan distance, according to one or more embodiments shown and described herein;

FIG. 15 schematically depicts the binary matrix that divides the projection of the hole side on the midsagittal plane into two regions, according to one or more embodiments shown and described herein;

FIG. 16 schematically depicts a two-part mold for constructing a physical cranial implant, according to one or more embodiments shown and described herein;

FIG. 17 schematically depicts a system for implementing computer and software based methods to utilize the methods of FIGS. 1-16, according to one or more embodiments shown and described herein;

FIG. 18A schematically depicts a portion of a graphical user interface for permitting user input and displaying information to the user, according to one or more embodiments shown and described herein;

FIG. 18B schematically depicts another portion of a graphical user interface for permitting user input and displaying information to the user, according to one or more embodiments shown and described herein; and

FIG. 19 depicts representative images of patient-specific cranial implants fitted into four cadaveric specimens, according to one or more embodiments shown and described herein;

### DETAILED DESCRIPTION

Reference will now be made in detail to various embodiments of methods, processing systems, and computer-readable mediums for the automated designing of patient-specific cranial implants, examples of which are illustrated in the accompanying drawings. Whenever possible, the same reference numerals will be used throughout the drawings to refer to the same or like parts.

As will be described in various embodiments of the present disclosure, methods, processing systems, and computer-readable mediums are described herein as utilizing an automated design process for patient-specific cranial implants (PSCIs). Application of various image processing algorithms, interpolation methods, computational geometry algorithms, and optimization approaches form the building blocks of the exemplary embodiments. These building blocks are based on the patient's data (i.e., Digital Imaging and Communications in Medicine (DICOM) data). The DICOM data includes images of the skull that are scanned after surgical removal of the skull flap. Moreover, the PSCI is custom designed using open-source routines that make the implant patient-specific and cost-effective.

The exemplary approaches for designing the PSCIs of the present disclosure involve, for example, the application of image processing algorithms to identify the mid-sagittal plane of the skull and mirror the pristine side of the skull to create the cranial implant for the cavity. Additionally, using a ray-tracing approach, an automated method is provided to identify the inside and outside surfaces of the skull. The inside and outside surfaces of the pristine side of the skull are used as the basis to form the inside and outside surfaces of the PSCI. This is computed by mirroring the pristine side to the hole-side followed by using a subtraction operation that outputs the PSCI geometry. A pixel encoding method is then utilized to align the inner and outer surfaces, which is important to generating an accurate PSCI. The PSCI thus designed is output in a stereolithography file format (STL) which can be 3D printed using an additive manufacturing apparatus and method, such as fused deposition modeling, for example. However, other additive manufacturing technologies may be used, such as binder jetting and laser sintering, for example.

Directional terms as used herein—for example up, down, right, left, front, back, top, bottom—are made only with reference to the figures as drawn and are not intended to imply ab solute orientation unless otherwise specified.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an,” and “the” include plural referents unless the context clearly dictates otherwise. Thus, for example, reference to “a” component includes aspects having two or more such components, unless the context clearly indicates otherwise.

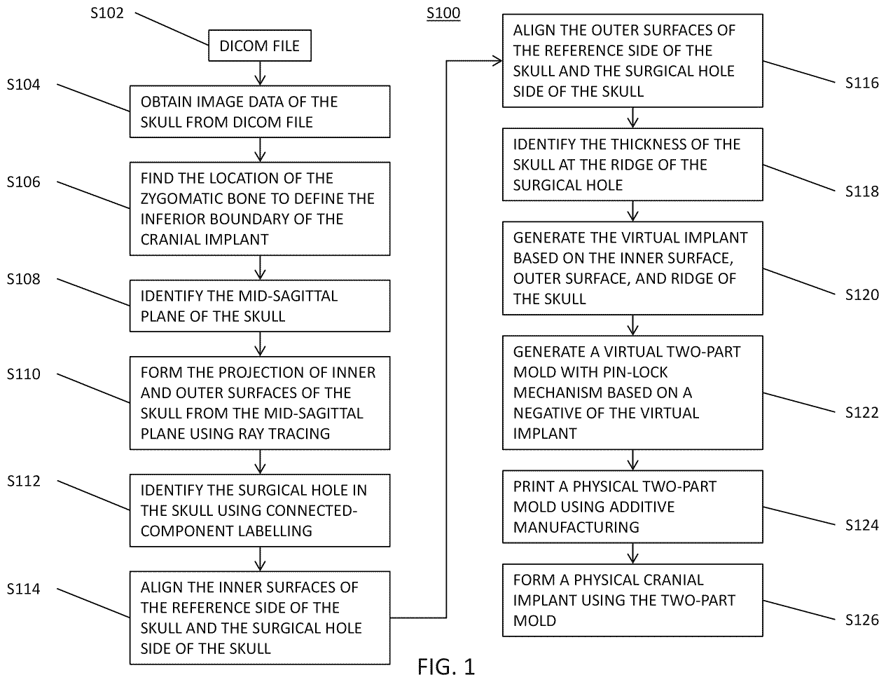

Referring to FIG. 1, an example method S100 for making a cranial implant is illustrated in accordance with embodiments of the present disclosure. The method S100 generally includes obtaining a DICOM file at S102. The DICOM file is generated after the patient's head is imaged using one of various modalities such as, for example, computed tomography (CT), magnetic resonance imaging (MRI), ultrasonography, and radio fluoroscopy. At S104, image data representative of the skull is obtained from the DICOM file such that a virtual model of the skull can be extracted from the image of the patient's head. Next, at S106, the location of the zygomatic bone is found on the virtual skull model to define the inferior boundary of the virtual cranial implant. The mid-sagittal plane of the virtual skull model is then identified at S108. At S110, projections of the inner and outer surfaces from the mid-sagittal plane of the virtual skull model are formed using ray tracing. The surgical hole in the skull is then identified using connected-component labelling at S112. Then, at S114, the inner surface of the reference side of the virtual skull model and the inner surface of the surgical hole side of the virtual skull model are aligned. After alignment of the inner surfaces, the outer surface of the reference side of the virtual skull model and the outer surface of the surgical hole side of the virtual skull model are aligned at S116. At S118, the thickness of the virtual skull model at the ridge of the surgical hole is identified. At S120, a virtual cranial implant is generated based on the inner surface, the outer surface, and the ridge of the virtual skull model. Next, at S122, a negative of the virtual cranial implant is formed and a virtual two-part mold is generated based on the negative. Then a physical two-part mold based on the virtual two-part mold is printed using an additive manufacturing apparatus at S124. Finally, at S126, a physical cranial implant is formed using the physical two-part mold. Each step S102-S126 of example method S100 will now be discussed in greater detail.

Method step S102 is directed to obtaining a DICOM file of the patient's head. A DICOM file is a standard adopted throughout the radiological field to securely exchange medical imaging data of patients. Various modalities such as CT scanning, MRI, ultrasonography, and radio fluoroscopy utilize the DICOM standard for image data transfer. Apart from the image data, a DICOM file may contain, for example, attributes such as patient name and ID, a data encoding scheme used for a certain scan, the imaging machine identification tags, and the like.

The DICOM images are one of the main attributes encoded into the DICOM file obtained at S102. They are images of a part of the body that is stored in the form of slices or layers. For example, a DICOM file of a head may contain numerous sub-files, each containing the image of a slice of the head. Using the pixel spacing and slice thickness attributes embedded within each slice, a complete 3D representation of the head can be assembled using all the individual slices. In this way, a DICOM file representation is similar to a sliced stereolithography (STL) file used in additive manufacturing. The example method S100 of the present disclosure operates on the image (i.e., pixel space) of each slice of the DICOM file.

Method step S104 is directed to obtaining image data from the DICOM file representative of the skull, such that a virtual model of the skull can be extracted from the image of the patient's head. The image data in a DICOM file is stored as an attribute called PixelData (pixel-data). The pixel-data is an image having a typical resolution of 512 by 512. Within the pixel-data attribute, pixel information may be stored in different formats such as, for example, integer values, float values, and/or RGB values. Additionally, the pixel-data can be encoded using common image compression standards, such as JPEG Baseline and RLE Lossless standards, for example. In step S104, the PyDicom library may be used to extract the pixel-data. PyDicom is a Python package configured to work with DICOM files such that a user can easily read and write these DICOM files into natural pythonic structures for easy manipulation. Upon extraction of the pixel-data, a separate library called the Grassroot DICOM or GDCM library may be used to read the compressed pixel-data in the form of an n-dimensional Numpy array. Since GDCM is native to the C++ programming language, the binaries for GDCM were built using CMake to obtain relevant python files. At this stage, the pixel-data is represented by integer values.

In order to obtain the pixel-data of the skull, and thus extract a virtual model of the skull from the image of the patient's head, thresholding is performed on the Numpy pixel arrays obtained using the GDCM library. It is noted that every type of tissue and bone absorbs radiation (X-ray and CT) differently, and the Hounsfield unit (HU) represents the coefficient of absorption of radiation. In the DICOM images, a single slice is represented by Hounsfield units. Thus, a single slice of a DICOM image contains HU values that form a grey-scale image. The HU values of interest belong to the bone or skull, which range from about 700 HU to about 2,000 HU. To extract the skull, all other values of HU that lie outside of this range are set at a zero threshold. This process is repeated for all the DICOM slices to extract the virtual skull model from the head.

Additional attributes for visualizing the virtual skull model include PixelSpacing, SliceThickness, Image Position (Patient), and Image Orientation (Patient). PixelSpacing is the distance between the centers of two adjacent pixels in both directions, represented by two numerical values. The Slice Thickness is the distance between two consecutive slices in the physical space of the patient. Image Position is the X, Y, and Z coordinates of the top left corner of the image, similar to how normal image matrices work. Image Orientation is the direction cosines of the first row and first column of the image matrix (or array depending on the programming language). Both Image Position and Image Orientation are specified for the patient during the imaging procedure.

With the virtual skull model having been extracted after method step S104, the location of the zygomatic bone can then be found on the virtual skull model to define the inferior boundary of the virtual cranial implant or the surgical hole at method step S106. The lower portion or inferior boundary of the surgical hole may not be well-defined due to irregularities in medical imaging (e.g., CT scanning), DICOM imaging, and/or HU values. This leads to an infeasible virtual cranial implant without a clear lower boundary. To solve this problem, the lower level or inferior boundary of the virtual cranial implant needs to be pre-defined.

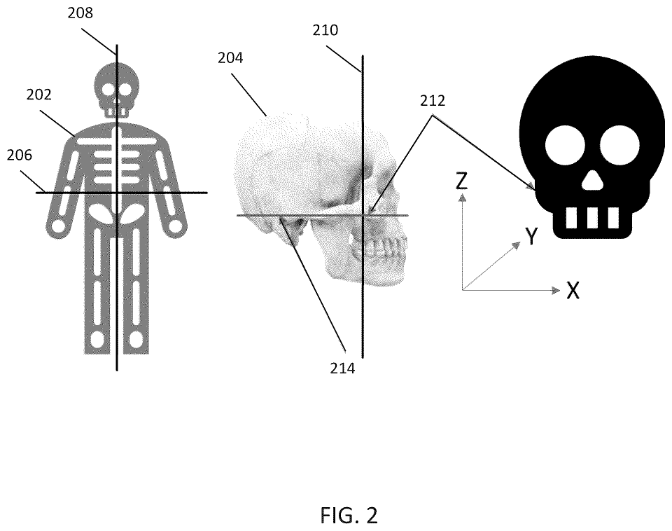

Terminology related to the locating of the zygomatic bone will now be discussed with reference to FIG. 2. In particular, FIG. 2 illustrates three planes of view from an anatomical point-of-view of the body 202 and the skull 204. These planes include the axial plane 206, sagittal plane 208, and coronal plane 210. The axial plane 206 is a horizontal plane dividing the body 202 into upper and lower parts. The sagittal plane 208 is a vertical plane dividing the body 202 into right and left sides. The coronal plane 210 is a vertical plane dividing the skull 204 into anterior and posterior portions. The Cartesian coordinate system, often called the reference coordinate system, is also shown in FIG. 2 and includes the X-axis traversing from right to left, the Y-axis traversing from front to back, and the Z-axis traversing from feet to head.

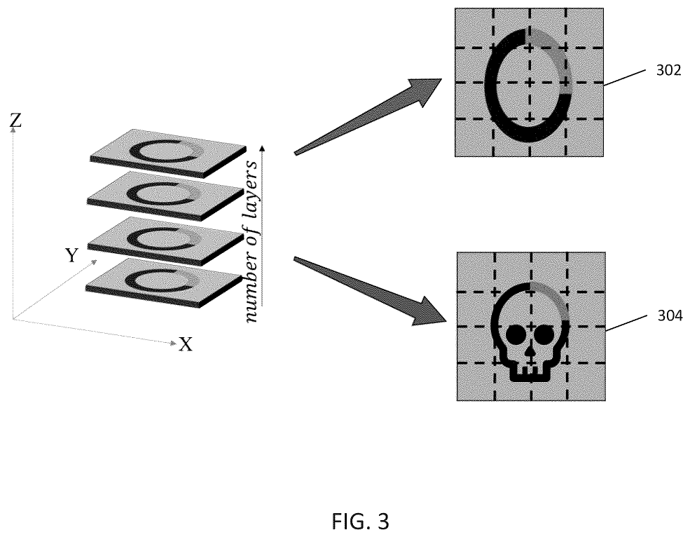

The inferior boundary 214 of the virtual cranial implant is assumed to be at the same level as the zygomatic bone 212 of the skull 204. The inferior boundary 214 is obtained using a combination of a ray-tracing approach and a variation of the bounding-box approach. Two assumptions are made to facilitate the calculation of the inferior boundary 214 of the virtual cranial implant. First, the zygomatic bone 212 can be detected by considering only the first half of the skull in the coronal plane 210 as the plane traverses from the front of the skull to the back, parallel to the XZ plane. Second, while traversing along the positive Y-axis, the zygomatic bone 212 forms the widest feature along the positive X-axis. In the axial plane 206, the DICOM slices are represented by a 3-dimensional matrix whose size can be defined as (512, 512, number of slices). The first two indices are the number of rows and columns in a single slice (pixel-data). In the coronal plane 210, parallel to the XZ plane or viewing along the positive Y-axis, the data is represented as (number of slices, 512, 512). For identifying the zygomatic bone 212 or inferior boundary 214, coronal representation is used. The matrix representation of the layer-wise DICOM file is illustrated in the middle left side illustration of FIG. 3. The upper right side illustration 302 of FIG. 3 is an axial view of a single DICOM slice that is parallel to the XY plane. The lower right side illustration 304 of FIG. 3 is coronal view of a single DICOM slice that is parallel to the XZ plane.

As shown in the lower right side illustration of FIG. 3, a bounding box 304 is constructed for each (number of slices×512) matrix in the coronal plane and is repeated 256 times. This is based on the earlier assumption that only the first half of the skull in the coronal plane is considered for detection of the zygomatic bone. The bounding box 302, 304 is a rectangle that includes all the non-zero pixels on a particular matrix/pixel space. The Y-level (in this case, the number of the slice index) where the pixel touches the bounding box 304 is recorded for 256 layers. The widest bounding box along the X direction is selected as the candidate and the Y-level corresponding to that layer is selected as the zygomatic level or inferior boundary of the virtual cranial implant.

An example sequence of steps for the detection of the zygomatic bone in method step S106 is described by the following pseudocode:

```
Identification of inferior boundary (zygomatic bone) of the cranial
implant
Input: DICOM files
Output: zygomatic bone level for the inferior boundary of the virtual
cranial implant and an updated DICOM date ignoring all the layers
below the zygomatic bone
For loop 1 all the layers from 1 to 256 (in the coronal view):
  For loop 2 all the slices from 1 to the total number of slices
    1. Find the maximum width of the bounding box
    2. Record the slice number that corresponds to the maximum width
  end for loop 2
end for loop 1
```

Zygomatic bone corresponds to the level (slice number) where there is the maximum value in the width of the bounding box
Updated DICOM data can be formed by ignoring all the slices below the zygomatic bone

After method step S106 discussed above, the mid-sagittal (MS) plane of the virtual skull model is identified at method step S108. As discussed in further detail below, this involves a mirroring operation conducted on the pixel data on the pristine or non-surgical (reference) side of the virtual skull model to match and populate the surgical hole-side of the virtual skull model. FIG. 4 shows the step-by-step approach for method step S108 to find the MS plane using a ‘folding method.’ In this step, the data on which the MS plane is to be calculated is stored as a 3-dimensional matrix of booleans (or binary format). The boolean matrix is constructed by assigning is to all non-zero pixels after the HU segmentation performed in method step S104 discussed above. Each pixel point on each DICOM slice is converted to a voxel whose size is the same as the slice thickness since the slice thickness is an embedded data attribute in the DICOM data. It is assumed that the normal to the MS plane is fixed along the X-axis, which means, voxel data is formed in such a way that each layer is parallel to the XY plane, as shown in the upper right side illustration 302 of FIG. 3. The MS plane is a plane parallel to the YZ plane whose location is fixed based on the maximum symmetry on either side of the skull. As the orientation of the plane is locked, the position of the MS plane can be calculated by folding the voxels about an arbitrary plane using a brute-force search of a finite space until the plane yields a maximum symmetricity metric. This symmetricity metric is calculated by finding an intersection volume of the pristine or reference side of the skull and the surgical hole side of the skull when one side overlaps the other after a mirroring operation about a fixed MS plane.

In FIG. 4, an example initial MS plane 402 is defined. The voxels to the left of the MS plane 402 are mirrored to the right. After the mirroring operation, the number of pixels that overlap is counted and assigned as the value for the symmetricity metric. The first iteration 400a of FIG. 4 does not have any overlap and thus the symmetricity metric value is zero. In the second iteration 400b, the MS plane 402 is moved and there are two overlapping voxels, thus the symmetricity metric value is 2. In the third iteration 400c, the MS plane 402 is moved again and there are 8 overlapping voxels, thus the symmetricity metric value is 8. This process is repeated until a maximum value for the symmetricity metric is achieved, which means that there is a maximum extent of overlap and that the location of the MS plane has been found.

An example sequence of steps for the identification of the MS plane in method step S108 is described by the following pseudocode:

```
Determining the location of the MS plane for given DICOM data
Input: Skull data in voxel format (3D matrix of Booleans)
Output: Location of the MS plane in voxel space
Variable symmetricity metric is set to 0 initially. It is an array that
measures symmetry metric for each candidate plane location
For loop all every possible plane location parallel to the YZ plane
  1. Get equally sliced matrices on each side of the assumed plane
  location
  2. Mirror one of the sides (matrix) to compare with the other side
  3. Calculate which pixels are bone-to-bone matches using a logical
  AND operation
  4. Store the number of matches in a particular plane location in
  symmetricity metric
end for loop
```

The location that yields the maximum value for the symmetricity metric is the MIDSAGITTAL Plane

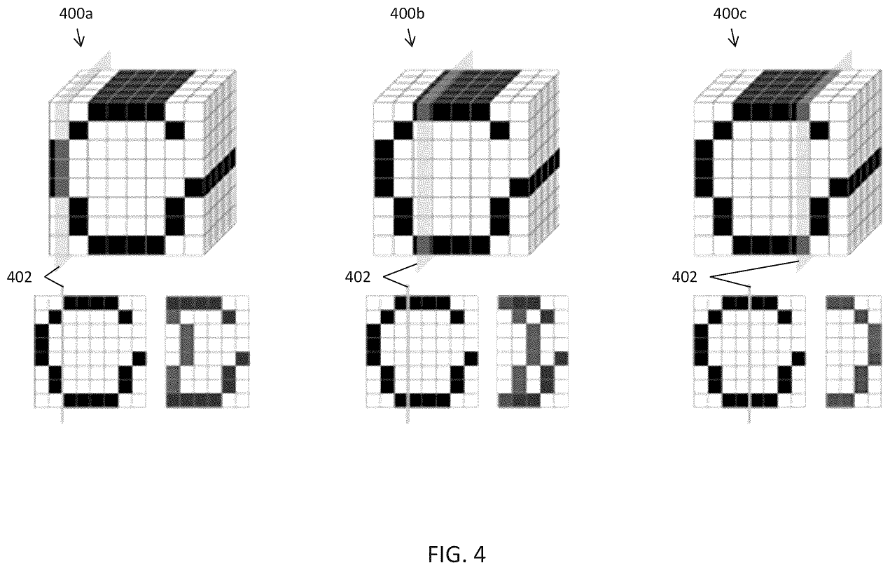

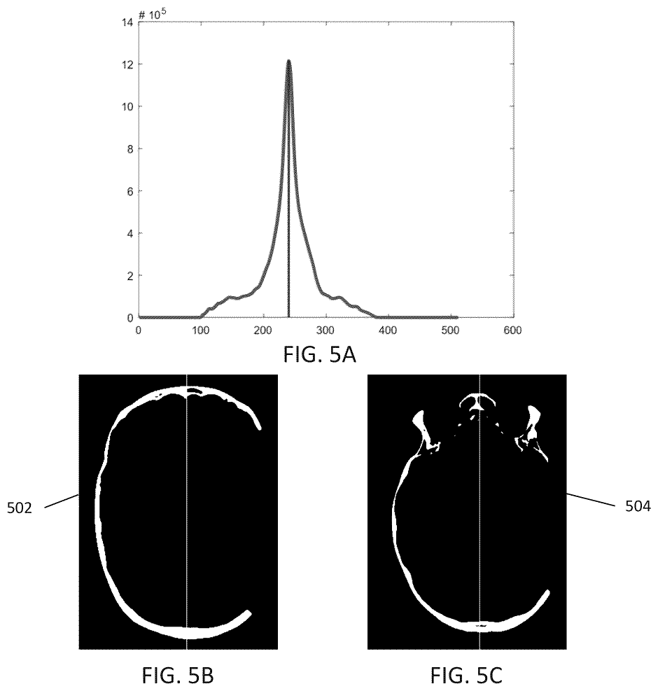

Thus, the pixel location where the symmetry metric is the highest is the location of the MS plane. An example is illustrated in FIG. 5 where the MS plane was found to be located at the 240^{th} position (i.e., between the 240ₜₕ and 241^{st} layer of voxels, as shown by the vertical line in the graph of FIG. 5). The location of the MS plane, as illustrated by the vertical line on layers 502, 504, on the test DICOM data is shown for two different layers 502, 504 at different heights.

After finding the MS plane at S108, the next step S110 is directed to finding the inner and outer sides of both halves of the skull from the MS plane. The voxel space of the virtual skull model includes a large number of voxels. The usual size of the DICOM slice image is 512 by 512 pixels. Thus, when each pixel space is converted to voxel format, this data becomes 512 by 512 by the number of layers/slices. Within each image slice, it is necessary to distinguish the inner and outer surface of the virtual skull model. The main reasons for finding the inner and outer surfaces are to identify the region where the surgical hole is situated in the virtual skull model and to differentiate the surgical hole-region and the rest of the skull. Current commercial software uses human intervention to identify the hole by permitting the user to manually select the regions that belong to the surgical hole. The drawback to this procedure is low accuracy due to human intervention. In accordance with one example embodiment of the present disclosure, method step S110 utilizes ray-tracing to identify the surgical hole at a voxel-level accuracy which cannot be achieved by manual methods.

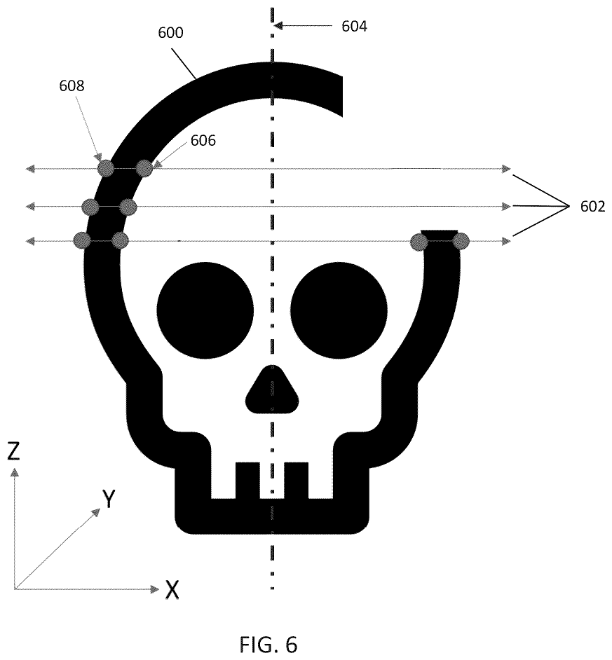

FIG. 6 illustrates the ray tracing of S110 to identify the inner and outer surfaces of the virtual skull model 600. To identify the inner and outer surfaces, a plurality of rays 602 are traced from the MS plane 604 towards either side of the skull (i.e., toward the pristine/reference side to the left and toward the surgical hole-side to the right). The rays 602 are kept parallel to the XY plane or normal to the MS plane. Along each row of voxels, the position of the first 606 and last solid voxel 608 is recorded, where a solid voxel is a voxel with a value of 1 and a void has a value of 0. The solid voxels that lie between the first 606 and last solid voxel 608 form the thickness of the virtual skull model. A separate record of the rows where no solid voxels are found is maintained. This record identifies the surgical hole that needs to be filled to form the virtual cranial implant and is explained below with respect to method step S112.

An example sequence of steps for the identification of the inner and outer surfaces of the virtual skull model using ray tracing in method step S110 is described by the following pseudocode:

```
Determining the inside and outside surface distances for the skull from
MS plane
Input: Skull data in voxel format, location of the MS plane
Output: Distance matrices for inner and outer surfaces of the skull on
both sides of the MS plane
Here a solid voxel has value 1 and empty voxel has value 0
For every value from 1 to 512 (the DICOM image resolution is
512 by 512 pixels)
  For every slice from 1 to the total number of slices
    1. Trace a ray on both sides of the MS plane
    2. Find the first and last solid voxel based on ray intersection
    3. Update distance matrices on both sides of the skull
    if there is no intersection (usually observed in the region where
      the hole is situated) note the distance value as 0 in the distance
      matrix
    end if statement
  end for loop
end for loop
```

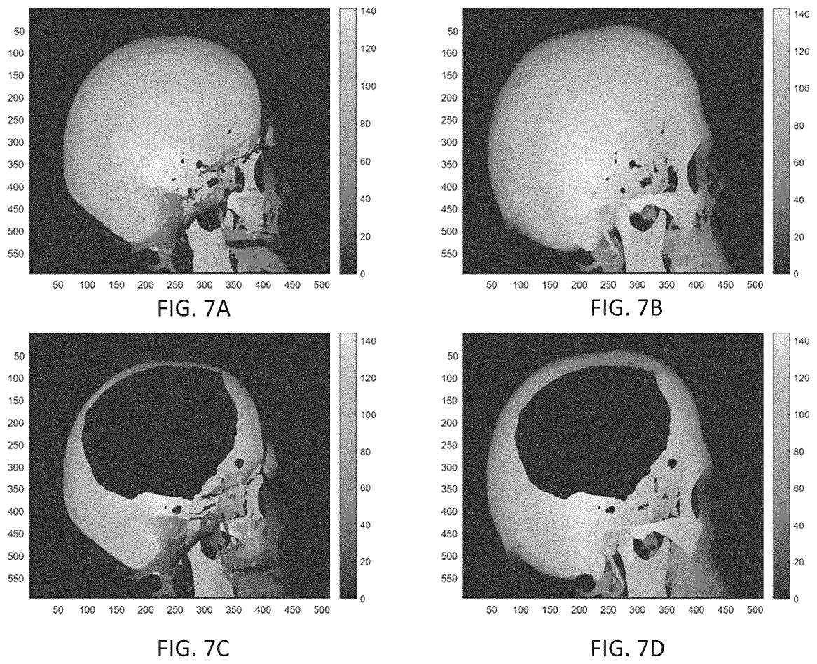

The voxels that lie between the first and last solid voxels in each row form the thickness of the virtual skull model. FIG. 7 shows the output after conducting the ray-tracing method of S110 on a sample DICOM data. The colormap plots of FIG. 7 show of the distance from the MS plane, where the darkest gray color represents the highest distance value. The values of the distance for each voxel row from either side of the MS plane are stored in a matrix. At the end of S110, there are four distance matrices. Each of the colormap plots in FIG. 7 are representative of one of the four distance matrices, where FIG. 7A illustrates the distance of the inner surface of the pristine or reference side of the virtual skull model from the MS plane, FIG. 7B illustrates the distance of the outer surface of the pristine or reference side of the virtual skull model from the MS plane, FIG. 7C illustrates the distance of the inner surface of the surgical hole-side of the virtual skull model from the MS plane, and FIG. 7D illustrates the distance of the outer surface of the surgical hole-side of the virtual skull model from the MS plane. The binary distance matrices, one for either side of the MS plane, points out the location (row and column index) of the surgical hole. A value of 1 in this matrix corresponds to solid, and a value of 0 corresponds to void. The distance matrices, two for each side, include the distance value from the MS plane to the first and last solid voxels in that row.

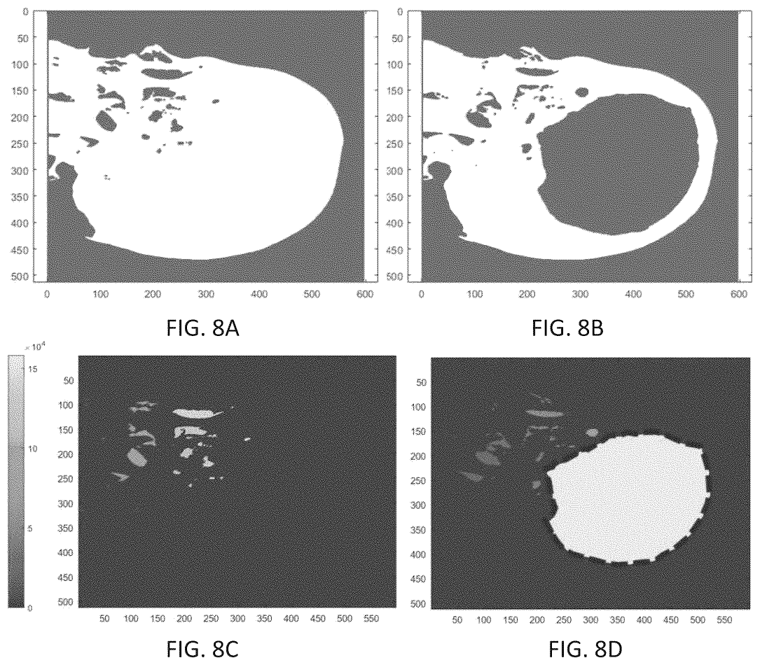

After finding the inner and outer surfaces of the virtual skull model at S110, the surgical hole is identified at S112. With reference to FIGS. 8A and 8B, the binary projections of the pristine or reference side of the virtual skull model and the surgical hole-side of the virtual skull model are shown on the MS plane, respectively. Based on observation, it is possible to identify the largest entity as the surgical hole in FIG. 8B. A connected-component analysis method is used to automate the process of identifying the largest entity or surgical hole. In the connected-component analysis, similar pixel values or pixel values that lie in a user-defined range are assigned a label. As shown in FIGS. 8C and 8D, an arbitrary empty cell is picked, and a flood filling algorithm is used to identify all of the other empty cells that are connected to it. All the connected cells are given the same label. The labeling scheme exhausts when there are no empty connected cells identified with a label. Next, another arbitrary and un-labeled cell is picked, and the same process is repeated until all connected cells are labeled as being in a cluster. Once all the pixels are assigned a label, a histogram of the labels can be created to count the number of pixels that share the same label. In this case, the largest connected-component corresponding to the surgical hole-region is expected to have the highest number of similar labels.

In the connected-component labeling (CCL) of method step S112, all the pixels are assigned a label. This includes the surrounding portion of the image that may not belong to the virtual skull model. The surrounding portion usually has a larger number of labels than the surgical hole. To prevent the risk of identifying the surrounding region of the image (due to the highest number of labels) as the surgical hole, all the components connected to the image border are neglected. All the disconnected regions within the pixel space are assigned different labels, as shown in FIGS. 8C and 8D. The surgical hole-region is identified by the dashed line in FIG. 8D based on the highest number of similar labels clustered together. Since the side of the skull (relative to the MS plane) that contains the surgical hole is known, the other side is used as the reference for determining the shape of the virtual cranial implant.

An example sequence of steps for the identification of the surgical hole of the virtual skull model in method step S112 is described by the following pseudocode:
Identifying the pixels corresponding to the surgical hole and the side of the skull it lies on

```
Input: Binary projection matrices (Images) of both sides of the skull
(Void = 0, solid = 1)
Output: 1) Region (pixel space) represented by the surgical hole,
    2) Side of the skull containing surgical hole
START
for each binary projection matrix (image)
    1. Ignore the pixels connected to the image boundary
    2. Identify the connected components using CCL
    3. Assign unique labels to each connected component identified
end for
END
```

The surgical hole is identified as the component with the highest number of labels
The matrix that has the surgical hole is identified as the surgical hole-side

After identifying the surgical hole at S112, the inner surfaces of the pristine or reference side of the virtual skull model and the surgical hole-side of the virtual skull model are aligned at S114. The inner surface of the reference side will form the inner surface of the virtual cranial implant. However, these surfaces will not align perfectly due to the non-symmetry of the skull geometry. The skull has an asymmetric geometry with a unique bisecting plane. Due to the organic nature of the geometry of the skull, certain adjustments are necessary to accurately align the inner surfaces for the virtual cranial implant. The four distance matrices obtained at method step S110 described above, along with the location of the surgical hole identified at method step S112, form the toolset required to align the inner surfaces. Thus, the method of S114 is operated in the pixel space.

To align the inner surfaces corresponding to the pristine or reference side to the surgical hole-side, an offset or correction value needs to be calculated at S114. The distance to the inside surface of the pristine side has to be adjusted by the offset or correction value for obtaining the correct alignment on the surgical hole side. The mathematical representation of this approach is shown in equations (1) and (2) below.

in_{ref}+in_{offset}=inₕₒₗₑ (1)

in_{offset}=inₕₒₗₑ−in_{ref} (2)

The in_{ref} and inₕₒₗₑ are matrices that represent the distance of each pixel from the MS plane, calculated at method step S110 described above. The in_{offset} matrix is the offset values that need to be calculated to align the inner surfaces of the reference side to the surgical hole-side. The output obtained using this approach is shown in FIGS. 7A-7D described above with reference to method step S110. One limiting factor to this approach is when there is an unexpected hole in the pristine or reference side, possibly due to CT scan parameters or HU thresholding. For such cases, the value of the offset will be set to zero temporarily during implementation until a complete matrix of offset values is obtained. The location of pixels where there is a hole in the reference side is recorded.

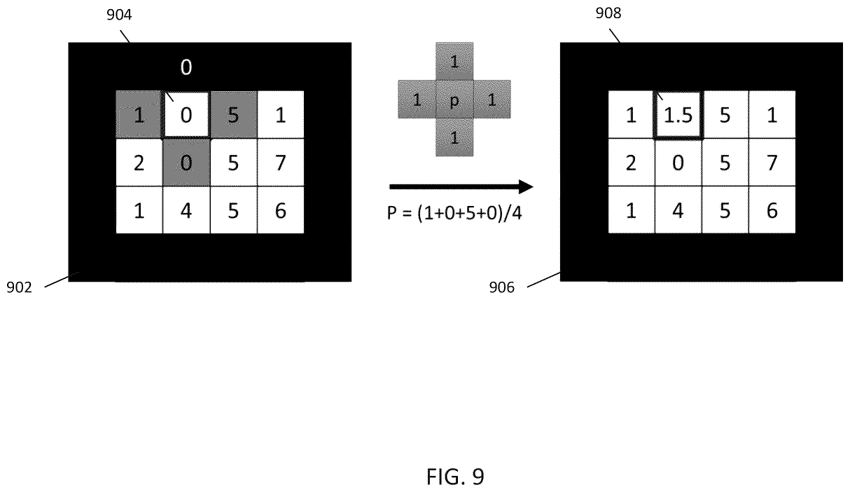

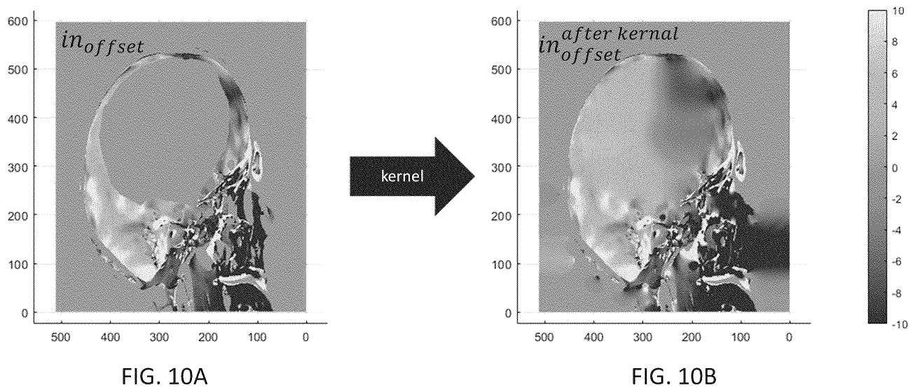

Once a complete offset matrix is obtained using equations (1) and (2) above, an average filtering approach using a 4-pixel kernel is utilized to fill the missing spots. The 4-pixel kernel 902, an example of which is illustrated in FIG. 9, is iteratively passed across each index (matrix place holder 904) and the average of the 4-pixel values (e.g., p=(1+0+5+0)/4) that are included in the kernel is substituted in the current pixel 908 of the example offset matrix 906. This process is repeated until all the indices with missing data are filled in the offset matrix. An example output result of this process is shown in FIG. 10, where the left side diagram represents the in offset matrix and the right side diagram represents the in offset matrix after the kernel is applied. The right side diagram of FIG. 10 is a smoothened version of the in offset matrix where all the regions with holes in the reference side of the virtual skull model are filled.

An example sequence of steps for obtaining the correct offset values for aligning the inside surfaces of the virtual skull model, along with the averaging operation for filling in missing regions in the offset matrix, in method step S114 is described by the following pseudocode:

```
Obtaining the offset values for aligning inside surfaces of the skull on
both sides of the MS plane
Input: the matrices that represent the distances of the inner surfaces of
the reference side and the surgical hole-side from the MS plane
Output: offset matrix for inside surfaces with corrected values
START
for every value from 1 to 512 (DICOM slice image)
  for all the slices from 1 to the total number of slices
    replace the pixel with the average of the four pixels that lie within
    the kernel
  end for
end for
END
```

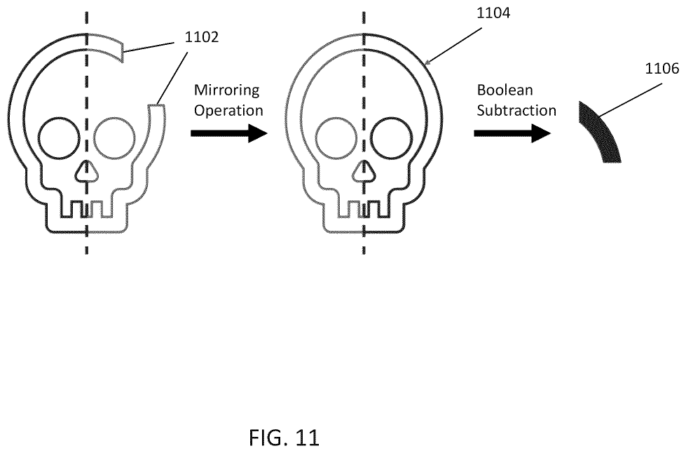

After aligning the inner surfaces of the reference side and the surgical hole-side of the virtual skull model at S114, the outer surfaces of the reference side and the surgical hole-side of the virtual skull model are aligned at S116. Method step S116 operates in the same manner as S114, however an additional step needs to be performed to properly align both outer surfaces. Mathematically, the outer surface of the virtual skull model can be identified in the pixel space by tracing a ray along each row of the pixel space (matrix) and recording the last non-zero pixel, as discussed above with respect to method step S110. In the physical space, the perimeter surface of the surgical hole (i.e., the ridge 1102 which defines the thickness of the skull) also counts as the outer surface, as illustrated in FIG. 11. FIG. 11 illustrates the mirroring operation to generate the aligned outer surface 1104 and the Boolean subtraction operation, discussed in further detail below, to form the virtual cranial implant 1106.

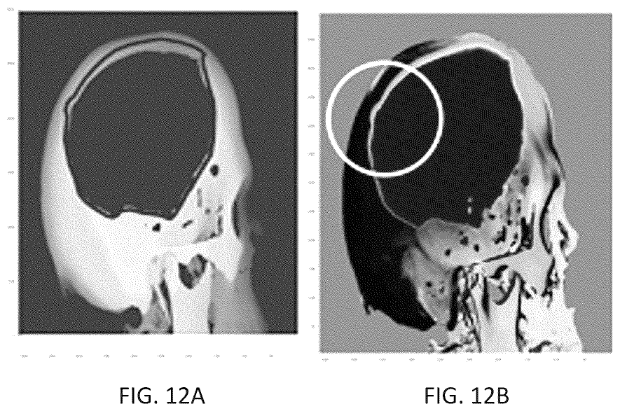

In order to properly align the outer surfaces, the precursor method step S118 is used to identify the ridge 1102 formed due to the surgical removal of the skull flap. In the left side diagram of FIG. 12, which illustrates the 2-D view of the virtual skull model when viewed from the MS plane, the perimeter portion outlined around the surgical hole indicates the ridge. The ridge is thus an outside surface in the physical space, but in the mathematical space relevant to the generation of the virtual cranial implant, steps to automatically identify the ridge are used for method step S118.

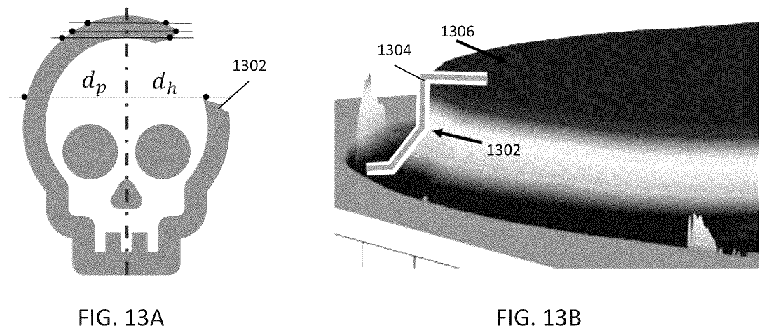

Referring to the left side illustration of FIG. 13, to identify the ridge's approximate location 1302 for method step S118, the difference between the distance from the MS plane to the pristine or reference outer surface (dₚ) and the distance from the MS plane to the outer surface of the surgical hole side (dₕ) is analyzed. Conceptually, the dilation of the inner edge of the ridge is tracked until the difference values plateau out. The reference outer surface and the surgical hole surface values are matrices that define the distance from the MS plane of each pixel in the reference outer surface and hole surface values. The right side image of FIG. 13 shows that the difference in the distance values (i.e., dₚ−dₕ) steadily decreases (as indicated by line 1304) when moving away from the surgical hole 1306. In other words, the ridge 1302 is the region where the steady decrease (line 1304) in the difference values stagnates. It can be concluded that the ridge ends when this steady decrease stagnates. Thus, by starting at the surgical hole and by traversing through pixels away from the surgical hole towards the outer surface of the skull, the difference values can be analyzed as a function of distance. When this minimum difference stagnates, it can be concluded that the outer perimeter surface of the surgical hole has been left behind, implying that the ridge has been identified.

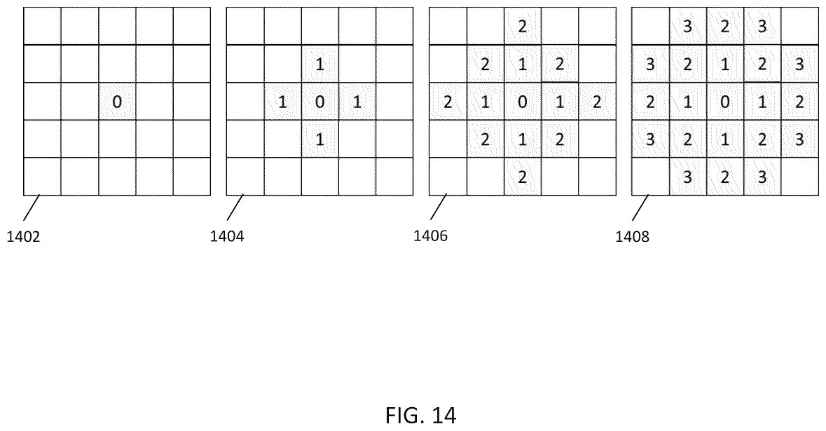

To analyze the difference values as part of method step S118, the first step is to calculate the distance from every pixel to the nearest pixel that has been identified as the surgical hole. The Manhattan distance is used because it is computationally more efficient to calculate in pixel space when compared to the Euclidean distance. A region-growing algorithm, an example of which is illustrated in FIG. 14, can be employed to calculate this efficiently. The start region consists of the pixels that belong to the surgical hole. In every iteration, i.e., as the region grows, the region in the previous iteration is dilated by 1 pixel. The start region 1402 that belongs to the surgical hole is assigned a Manhattan distance of 0. The new pixels thus formed due to the first dilation 1404 are assigned a Manhattan distance value of 1. The pixels covered by the new region in the second dilation 1406 are assigned a Manhattan distance value of 2. The pixels covered by the new region in the third dilation 1408 are assigned a Manhattan distance value of 3. This process is continued until all the pixels, except the start region belonging to the surgical hole, are assigned a particular Manhattan distance value.

Once all the pixels are assigned a Manhattan distance, an iterative procedure is used in method step S118 to analyze the difference values (difference in the distance values from the surgical hole side and the reference side) in the order of the Manhattan distance values that were previously assigned. For each pixel, a neighboring pixel that is closer to the surgical hole is checked to see if the difference value has decreased. If the difference value has decreased, the next pixel is chosen. If not, a stagnation counter, a temporary variable, is incremented. A stagnation counter is a variable that allows stagnation for a certain number of times. The stagnation counter is allowed to increment until the decreasing trend has stopped. A value of 3 has been chosen arbitrarily for the upper bound of the stagnation counter beyond which the iteration would stop. In other words, the iteration will allow stagnation three times after which it will stop the search. This indicates that the ridge has been identified and the ridge can be subsequently used to construct the final virtual cranial implant.

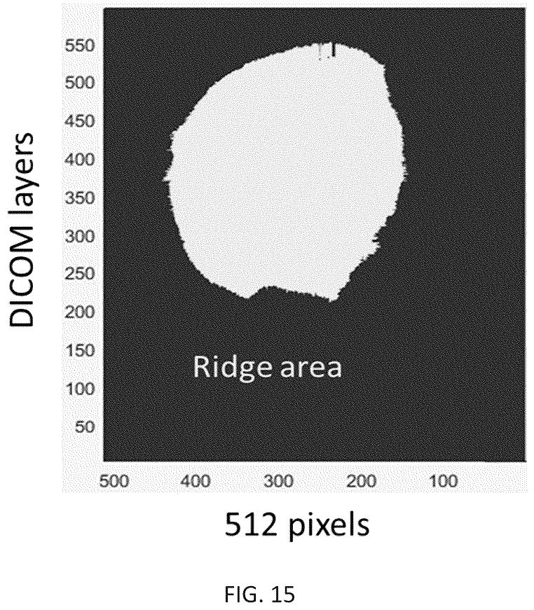

The ridge is defined using a binary matrix that divides the projection of the surgical hole-side on the MS plane into two regions. The hole and ridge form the first region as shown by the light gray color of FIG. 15. The rest of the space forms the second region as shown by the dark gray color of FIG. 15. Finally, to align the outer surfaces at method step S116, the same procedure is used as the inner surface alignment discussed above with respect to method step S114.

After aligning the outer surfaces of the reference side and the surgical hole-side of the virtual skull model at S116, and after identifying the ridge at S118, the virtual cranial implant can be generated at S120. At the end of method steps S116 and S118, four pieces of information are available that are required for the alignment of surfaces, including the inside and outside surfaces of the reference side and the offset matrices called in_{offset} and out_{offset}. Thus, the inner and outer surfaces of the virtual cranial implant are known, along with the adjustments that have to be made to align the virtual cranial implant with the surgical hole. However, the material inside the virtual cranial implant is unknown. In this regard, method step S120 utilizes a voxel-based approach to construct the virtual cranial implant.

To begin the process of generating a voxelized virtual cranial implant, a fully void voxelization is initialized for the entire virtual skull model. Next, the offset values are added to the reference sides to create new reference sides that will properly align with the inner and outer surfaces of the surgical hole-side. The new reference sides for the inner and outer surfaces of the surgical hole-side are obtained using equations (3) and (4) below:

in_{ref}+in_{offset}=aligned_in_{ref} (3)

out_{ref}+out_{offset}=aligned_out_{ref} (4)

Subsequently, four matrices are defined that can be used to construct the virtual cranial implant, including aligned_in_{ref}, aligned_out_{ref}, outₕₒₗₑ, and ridge_matrix. The aligned_in_{ref} and aligned_out_{ref} matrices can be obtained using equations (3) and (4) above. The out hole matrix is the distance of the outer surface of the surgical hole-side of the skull from the MS plane, as discussed in method step S110 described above. The ridge_matrix is obtained as a result of method step S118. Boolean True values can be assigned to all the voxels that lie within the range of the aligned_in_{ref} and aligned_out_{ref} matrices (i.e., all the voxels that lie between the inner and outer surfaces are assigned as solid. This operation is only carried out for all the pixels that have a False value in the ridge_matrix (i.e., the area belonging to the hole and the ridge). Any pixels beyond the range mandated by the outₕₒₗₑ matrix are marked False.

For method step S120, an example sequence of steps for the generation of the voxelized virtual cranial implant using the four matrices described above is described by the following pseudocode:

```
Virtual Cranial Implant Generation
Input:
1. The distance matrix that shows the aligned inner surface in the
reference side
2. The distance matrix that shows the aligned outer surface in the
reference side
3. The matrix that defines the surgical hole
4. The matrix that defines the location of the ridge from the MS plane
Output: Virtual cranial implant constructed in voxel space
for all the values that lie from 1 to the width of the matrix (512)
  for all the values that lie from 1 to the height of the matrix (number
  of slices)
    start: Full void voxelization
    if starting voxel lies in the position of the ridge matrix
      1. Set every voxel in that row which is in range between the
      aligned inner and outer surfaces equal to True (solid)
      2. Set every voxel in that row which is outside the range
      between aligned inner and outer surfaces equal to False (void)
    End if
  End for
End for
```

All the solid voxels in the surgical hole-side are subtracted to form the virtual cranial implant

In accordance with some embodiments of the present disclosure, the generation of the voxelized virtual cranial implant at method step S120 may include constructing an STL file of the implant that can be 3D printed with an additive manufacturing apparatus. In this regard, a marching cubes algorithm is used to construct the STL file of the implant. However, it is noted that the conversion of the voxelized virtual cranial implant to the STL file can be carried out using any methods that can create iso-surfaces necessary for STL file information. The resultant STL file is passed through a smoothing filter to smoothen out the jagged edges, often a by-product of the marching cubes output. This STL file can be 3D printed using FDA-approved implant materials for direct use. However, subsequent sterilization and the 3D printable implant material costs may hinder this approach in some scenarios. Thus, a two-part mold manufactured with easy-to-access 3D printable materials becomes a viable choice.

Once the voxelized virtual cranial implant is generated at S120, the method continues at S122 to generate a virtual two-part mold based on a negative of the virtual cranial implant. The negative of the virtual cranial implant consists of the two-part mold, with one part for each of the inner and outer surfaces of the virtual cranial implant. To generate one-half of the virtual two-part mold, a ray is traced from the lowest point of the inner-outer surface range until the virtual cranial implant is hit. All voxels before the virtual cranial implant regions are recorded with a value of 1 or solid, implying a solid implant. For the second half of the virtual two-part mold, ray tracing is repeated from the highest point of the inner-outer surface range to the outer side of the virtual cranial implant. Again, all voxels before the virtual cranial implant regions are recorded with a value of 1 or solid. A virtual two-part mold is thus provided with a gap in the middle at the end of method step S122.

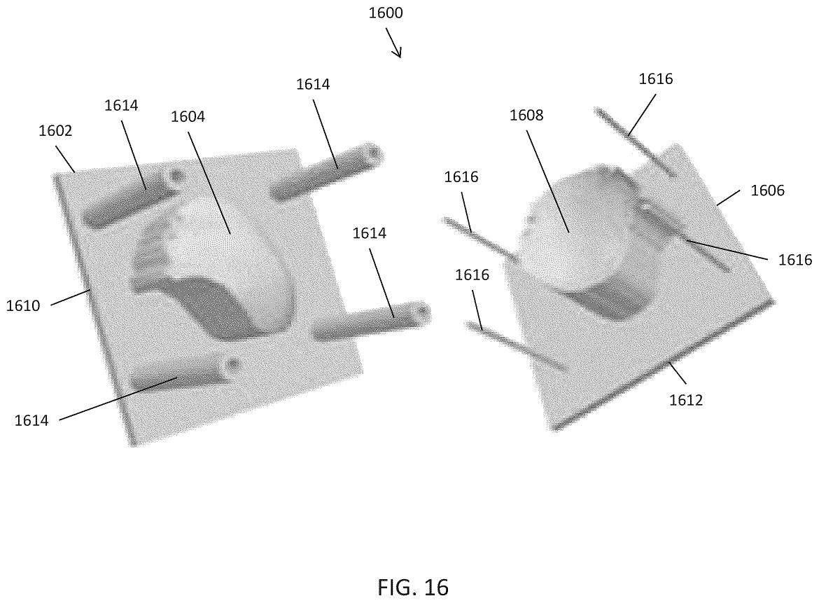

Additionally, to provide a non-slipping, error-free two-part mold assembly, the two halves of the mold may be embedded on a pin-lock mechanism that ensures proper fit of the two halves during the physical cranial implant manufacture at method step S126 discussed below. The pin-lock mechanism may be automatically formed when the virtual two-part mold is generated in method step S122. In FIG. 16, an example two-part mold 1600 is illustrated in accordance with embodiments of the present disclosure. The two-part mold 1600 includes a first mold 1602 with a forming surface 1604 that defines the inner surface of the physical cranial implant when the physical cranial implant is formed and a second mold 1606 with a forming surface 1608 that defines the outer surface of the physical cranial implant when the physical cranial implant is formed. The pin-lock mechanism includes a first base 1610 that supports the forming surface 1604 of the first mold 1602 and a second base 1612 that supports the forming surface 1608 of the second mold 1606. The first base 1610 also supports a plurality of tubular pin receiving members 1614 extending outward from the first base 1610, while the second base 1612 also supports a plurality of pins 1616 extending outward from the second base 1612.

Once the forming surfaces 1604 and 1608 are aligned such that the inner and outer surfaces of the physical cranial implant will be properly formed, the plurality of pin receiving members 1614 are configured to receive the plurality of pins 1616 and thereby secure the two molds 1602, 1606 together when it is desired to form the physical cranial implant. The plurality of pins 1616 can have an adjustable length that is selected to be greater than the length of the plurality of pin receiving members 1614 such that a gap is formed between the forming surfaces 1604, 1608 when the two molds 1602, 1606 are secured together via the engagement of the plurality of pins 1616 within the plurality of pin receiving members 1614. In this regard, the length of the plurality of pins 1616 can be predefined by a user such that a thickness of the gap between the forming surfaces 1604, 1608 can be controlled. This gap thickness defines the thickness of the physical cranial implant formed using the two-part mold 1600. In some embodiments, a gap thickness of about 4 mm is recommended.

After the virtual two-part mold and pin-lock mechanism are generated in method step S122, a physical two-part mold can be printed using additive manufacturing at method step S124. In order to be able to print the physical two-part mold, an STL file of the virtual two-part mold must first be constructed. The STL file may be constructed using a marching cubes algorithm. However, it should be noted that the conversion to the STL file can be carried out using any method that can create iso-surfaces necessary for STL file information. The resultant STL file is passed through a smoothing filter to smoothen out the jagged edges. This STL file can then be 3D printed using common additive manufacturing apparatuses and common 3D printing materials, such as poly-lactic acid (PLA), for example.

In accordance with some embodiments of the present disclosure, since the two-part mold is generated based on voxels and STL model processing, a slight tolerance may be manually added to the final two-part mold design to account for any mathematical over-estimation of the implant and mold size. This tolerance may be incorporated by scaling down the virtual cranial implant generated after method step S120 uniformly in all directions. In some particular embodiments, the virtual cranial implant is scaled down by a user-defined scale factor of about 3%. In this regard, the STL models of the virtual cranial implant and the physical implants manufactured using the two-part molds were observed to fit better when shrunk by the user-defined scale factor and tested in cadaver models, as discussed in further detail below. The scaling operation is carried out to account for the dimensional tolerances and surface roughness expected in 3D printed outputs.

Once the physical two-part mold is printed at method step S124, the physical cranial implant can be formed at method step S126. To form the physical cranial implant, a biocompatible material, such as PMMA bone cement, for example, can be selected and prepared according to supplier instructions and allowed to set until reaching a plastic, clay-like consistency. Once the desired consistency is reached, the biocompatible material may be first inserted into a sterile plastic sleeve typically provided by the biomaterial supplier. The biocompatible material is then flattened in the sterile plastic sleeve and pressed between the two parts of the mold. The biocompatible material is allowed to fully set between the two parts of the mold. Once fully set, a physical cranial implant is formed from the biocompatible material which can be subsequently secured into the cranial defect (i.e., the surgical hole) using appropriate fasteners, such as titanium screws and miniplates, for example. In addition, prior to being secured in the cranial defect, the physical cranial implant may be manually modified by a user as needed to ensure an optimal fit.

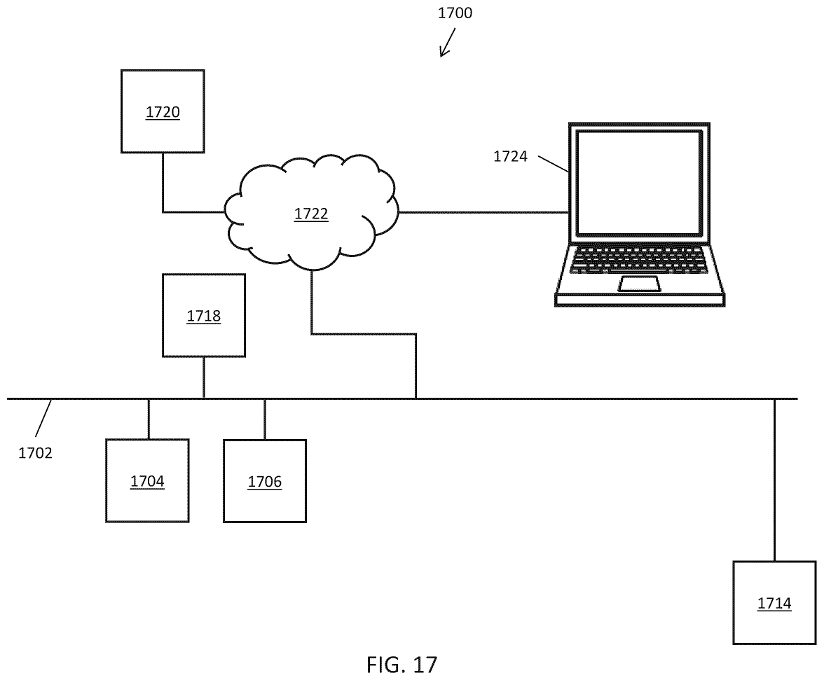

Referring to FIG. 17, a system 1700 for implementing a computer and software-based method, such as method S100 described herein and as shown in FIGS. 1-16, may be implemented along with using a graphical user interface (GUI) displaying, for example, visualizations of the skull (with the defect or surgical hole), the virtual cranial implant, and also the two-part mold. The GUI may be accessible on a display at a user workstation (e.g., a computing device 1724), for example. The system 1700 includes a communication path 1702, one or more processors 1704, a memory component 1706, for example, a storage or database 1714, a network interface hardware 1718, a network 1722, a server 1720, and at least one computing device 1724. The various components of the system 1700 and the interaction thereof will be described in detail below.

In some embodiments, the system 1700 is implemented using a wide area network (WAN) or network 1722, such as an intranet or the Internet. The computing device 1724 may include digital systems and other devices permitting connection to and navigation of the network. Other system 1700 variations allowing for communication between various geographically diverse components are possible. The lines depicted in FIG. 17 indicate communication rather than physical connections between the various components.

As noted above, the system 1700 includes the communication path 1702. The communication path 1702 may be formed from any medium that is capable of transmitting a signal such as, for example, conductive wires, conductive traces, optical waveguides, or the like, or from a combination of mediums capable of transmitting signals. Moreover, in some embodiments, the communication path may facilitate the transmission of wireless signals, such as Wi-Fi, Bluetooth, and the like. Additionally, the communication path may comprise a bus, such as for example ISA, EISA, PCI, PCI Express, USB, Serial ATA or FireWire or the like. The communication path 1702 communicatively couples the various components of the system 1700. As used herein, the term “communicatively coupled” means that coupled components are capable of exchanging data signals with one another such as, for example, electrical signals via conductive medium, electromagnetic signals via air, optical signals via optical waveguides, and the like.

As noted above, the system 1700 includes the processor 1704. The processor 1704 can be any device capable of executing machine readable instructions. Accordingly, the processor 1704 may be a controller, an integrated circuit, a microchip, a computer, or any other computing device. The processor 1704 is communicatively coupled to the other components of the system 1700 by the communication path 1702. Accordingly, the communication path 1702 may communicatively couple any number of processors with one another, and allow the modules coupled to the communication path 1702 to operate in a distributed computing environment. Specifically, each of the modules can operate as a node that may send and/or receive data.

As noted above, the system 1700 includes the memory component 1706 which is coupled to the communication path 1702 and communicatively coupled to the processor 1704. The memory component 1706 may be a non-transitory computer readable medium or non-transitory computer readable memory and may be configured as a nonvolatile or volatile computer readable medium. The memory component 1706 may comprise RAM, ROM, flash memories, hard drives, or any device capable of storing machine-readable instructions such that the machine-readable instructions can be accessed and executed by the processor 1704. The machine-readable instructions may comprise logic or algorithm(s) written in any programming language such as, for example, machine language that may be directly executed by the processor, or assembly language, object-oriented programming (OOP), scripting languages, microcode, etc., that may be compiled or assembled into machine readable instructions and stored on the memory component 1706. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. In embodiments, the system 1700 may include the processor 1704 communicatively coupled to the memory component 1706 that stores instructions that, when executed by the processor 1704, cause the processor to perform one or more functions as described herein.

Method S100 described herein may be implemented in one or more computer programs that may be executable on a programmable system 1700 including at least one programmable processor 1704 coupled to receive data and instructions from, and to transmit data and instructions to, a data storage system, such as memory component 1706, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Still referring to FIG. 17, as noted above, the system 1700 comprises the display such as a GUI on a screen of the computing device 1724. The display on the screen of the computing device 1724 is coupled to the communication path 1702 and communicatively coupled to the processor 1704. Accordingly, the communication path 1702 communicatively couples the display to other modules of the system 1700. The display can include any medium capable of transmitting an optical output such as, for example, a cathode ray tube, light emitting diodes, a liquid crystal display, a plasma display, or the like. Additionally, it is noted that the display or the smart device 1724 can include at least one of the processor 1704 and the memory component 1706. While the system 1700 is illustrated as a single, integrated system in FIG. 17, in other embodiments, the systems can be independent systems.

The system 1700 includes the network interface hardware 1718 for communicatively coupling the system 1700 with a computer network such as network 1722. The network interface hardware 1718 is coupled to the communication path 1702 such that the communication path 1702 communicatively couples the network interface hardware 1718 to other modules of the system 1700. The network interface hardware 1718 can be any device capable of transmitting and/or receiving data via a wireless network. Accordingly, the network interface hardware 1718 can include a communication transceiver for sending and/or receiving data according to any wireless communication standard. For example, the network interface hardware 1718 can include a chipset (e.g., antenna, processors, machine readable instructions, etc.) to communicate over wired and/or wireless computer networks such as, for example, wireless fidelity (Wi-Fi), WiMax, Bluetooth, IrDA, Wireless USB, Z-Wave, ZigBee, or the like.

Still referring to FIG. 17, data from various applications running on the computing device 1724 can be provided from the computing device 1724 to the system 1700 via the network interface hardware 1718. The computing device 1724 can be any device having hardware (e.g., chipsets, processors, memory, etc.) for communicatively coupling with the network interface hardware 1718 and a network 1722. Specifically, the computing device 1724 can include an input device having an antenna for communicating over one or more of the wireless computer networks described above.

The network 1722 can include any wired and/or wireless network such as, for example, wide area networks, metropolitan area networks, the Internet, an Intranet, satellite networks, or the like. Accordingly, the network 1722 can be utilized as a wireless access point by the computing device 1724 to access one or more servers (e.g., a server 1720). The server 1720 and any additional servers generally include processors, memory, and chipset for delivering resources via the network 1722. Resources can include providing, for example, processing, storage, software, and information from the server 1722 to the system 1700 via the network 1722. Additionally, it is noted that the server 1720 and any additional servers can share resources with one another over the network 1722 such as, for example, via the wired portion of the network, the wireless portion of the network, or combinations thereof

In embodiments, the system 1700 may include a processor 1704 and a non-transitory computer-readable memory 1706 communicatively coupled to the processor 1704. The memory 1706 may store instructions that, when executed by the processor 1704, cause the processor 1704 to follow one or more functions, such those set forth in blocks S102-S122 of the process S100 described above and illustrated in FIG. 1.

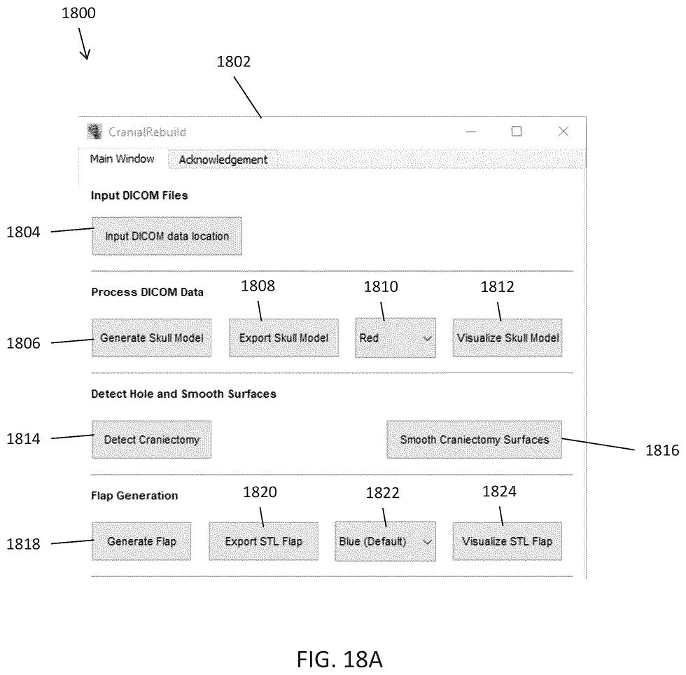

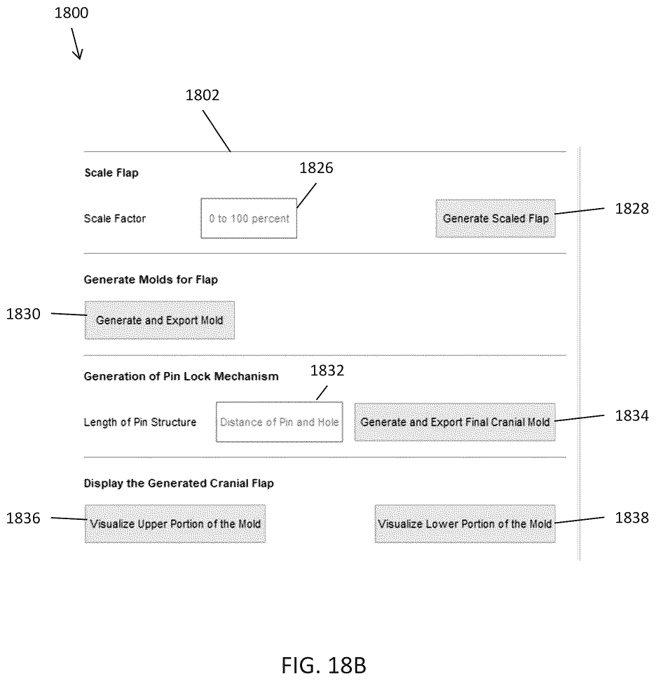

Referring to FIGS. 18A and 18B, a GUI 1800 in accordance with one embodiment of the present disclosure is illustrated. The GUI 1800 generally enables users to import the input DICOM files, visualize the skull (with the defect or surgical hole), the virtual cranial implant, and also the two-part mold, for example. Additionally, to provide the users with more control on the fit of the cranial implant on the skull, the scaling factor as mentioned before has been set as a user-defined parameter in the GUI 1800. This GUI 1800 provides users the opportunity to review the output from each intermediate step of method S100 described above, starting from the DICOM input file and skull generation to the final two-part mold generation. The GUI 1800 may be accessible on a display at a user workstation (e.g., computing device 1724 in FIG. 17), for example.

The GUI 1800 may be displayed when an application or software containing machine-readable instructions for implementing method S100 is launched on a computer device (e.g., computing device 1724 in FIG. 17). Thus, GUI 1800 may be displayed in response to a user request to display the GUI. For example, a user may launch application or software by selecting an application or software icon displayed on a display device of an associated computing device. Generally, the GUI 1800 permits user input and displays information to the user during the implementation of method S100.

In FIG. 18A, a display screen 1802 of GUI 1800 is illustrated. The display screen 1802 includes an “Input DICOM files” section including a button 1804 that is user-selectable to input DICOM files. Upon selection of the input DICOM file button 1804, a separate display screen may appear to select a desired DICOM file. Button 1804 is thus configured to cause an associated processor (such as processor 1704 of system 1700) to implement method step S102 described above.

The display screen 1802 further includes a “Process DICOM data” section including four buttons 1806, 1808, 1810, 1812 which are selectable by a user to utilize the data stored in the DICOM file which was previously input using button 1804. Button 1806 provides an option to “Generate Skull Model.” Upon selection of button 1806, the processor may be instructed to implement method step S104 and image data related to the skull is obtained. Moreover, selection of button 1806 may also cause the processor to implement method steps S106-S110 and S114-S118 described above. Button 1808 provides an option to “Export Skull Model.” Upon selection of button 1808, a separate display screen may appear to select various parameters for exporting the skull model, such as exporting to a new file type, for example. Button 1810 provides an option to select a color for the skull model. Button 1812 provides an option to “Visualize Skull Model.” Selection of button 1812 may instruct the processor to display a 3D image of the skull model in display screen 1802 or a separate display screen.

The display screen 1802 also includes a “Detect Hole and Smooth Surfaces” section including two buttons 1814 and 1816. Button 1814 provides an option to “Detect Craniectomy.” It is noted that the term craniectomy can also be described as the surgical hole. Thus, selecting button 1814 causes the processor to implement method step S112 described above. Button 1816 provides an option to “Smooth Craniectomy Surfaces.” Selection of button 1816 may cause the processor to smoothen undesirable surface features of the craniectomy such as jagged edges, for example.

The display screen 1802 further includes a “Flap Generation” section including four buttons 1818, 1820, 1822, and 1824. It is noted that “Flap Generation” refers to the generation of the virtual cranial implant. Button 1818 provides an option to “Generate Flap.” Upon selection of button 1818, the processor may be instructed to implement method step S120 described above. Button 1820 provides an option to “Export STL Flap.” Selection of button 1820 may cause the processor to generate an STL file of the virtual cranial implant which can subsequently be used to print a physical cranial implant using an additive manufacturing apparatus in accordance with some embodiments described above. Button 1822 provides an option to select a color for the virtual cranial implant. Button 1824 provides an option to “Visualize STL Flap.” Selection of button 1824 may instruct the processor to display a 3D image of the virtual cranial implant in display screen 1802 or a separate display screen.

Referring now to FIG. 18B, the display screen 1802 continues and includes a “Scale Flap” section that includes a “Scale Factor” box 1826 and a “Generate Scaled Flap” button 1828. Scale Factor box 1826 permits a user to enter a numerical value of between 0 and 100 percent. As such, when button 1828 is selected, the processor may generate a scaled down version of the virtual cranial implant based on the value entered in box 1826. For example, a value of 97% may be input into box 1826 such that the virtual cranial implant is uniformly reduced in size by 3%.

The display screen 1802 also includes a “Generate Molds for Flap” section including button 1830 that provides an option to “Generate and Export Mold.” Upon selection of button 1830, the processor may be instructed to implement method step S122 described above such that a virtual two-part mold is generated.

Next, the display screen 1802 includes a “Generation of Pin Lock Mechanism” section that includes a “Distance of Pin and Hole” box 1832 and a “Generate and Export Final Cranial Mold” button 1834. Box 1832 permits a user to enter a numerical value corresponding to a length of the pin structure (e.g., plurality of pins 1616 discussed above with reference to FIG. 16). As such, when button 1834 is selected, the processor may generate a virtual two-part mold containing pins having the length specified in box 1832. Moreover, the remaining structures of the pin-lock mechanism, as discussed above, are generated.

Finally, the display screen 1802 includes a “Display the Generated Cranial Flap” section that includes a “Visualize Upper Portion of the Mold” button 1836 and a “Visualize Lower Portion of the Mold” button 1838. Selection of button 1836 may instruct the processor to display a 3D image of the upper portion of the virtual two-part mold (e.g., first mold 1602 discussed above with reference to FIG. 16) in display screen 1802 or a separate display screen. Selection of button 1838 may instruct the processor to display a 3D image of the lower portion of the virtual two-part mold (e.g., second mold 1606 discussed above with reference to FIG. 16) in display screen 1802 or a separate display screen.

### Example

Method S100 as described above was implemented as a fully automated computer program called “CranialRebuild.” CranialRebuild was launched on a computing device including a processing system and the processing system was instructed to operate in accordance with the user defined parameters and selectable options presented through use of the GUI 1800 described above. CranialRebuild was subsequently evaluated in four cadaveric specimens. Decompressive craniectomy was performed in the standard fashion on four cadaveric heads. Thin-cut CT imaging was subsequently obtained with positioning for optimal axial symmetry. The resulting DICOM images were processed through CranialRebuild program, which produced the STL files to be used for 3D printing with an additive manufacturing apparatus. All two-part molds were printed using a Stratasys F370 Industrial Fused Deposition Modeling (FDM) Printer with 1.75 mm PLA filament at 0.254 mm layer thickness, 0.4 mm nozzle diameter, 30% infill density, and 50 mm/s printing speed.

Following attainment of the two-part mold from the 3D printers, PMMA bone cement was prepared according to supplier instructions and allowed to set until reaching a plastic, clay-like consistency. In this state, it was inserted into the sterile plastic sleeve provided in the PMMA kit, flattened, and pressed between the two parts of the mold. The plastic sleeve and PLA mold are able to withstand the heat dissipated from the exothermic reaction of the PMMA setting. Once set, the cranial implant edges underwent modification as needed for optimal fit.

The cranial implant was subsequently secured into the cranial defect (surgical hole) using titanium screws and miniplates. For purposes of assessment of hermetic precision, a 5-point visual analogue scale (VAS) was utilized for grading (see Table 1 below). The grading scale evaluated final cosmesis, as well the degree of modification required in the event optimal cosmesis could be achieved.

TABLE 1
5-point visual analogue scale utilized to evaluate
the fit and cosmesis of the cranial implants.

| | |
|---|---|
| 5 | Perfect implant fit; optimal cosmesis can be achieved without post- |
| | processing modification. |
| 4 | Excellent implant fit; optimal cosmesis can be achieved with minimal |
| | post-processing modification. |
| 3 | Good implant fit; optimal cosmesis can be achieved with moderate |
| | post-processing modification. |
| 2 | Average implant fit; optimal cosmesis can be achieved with |
| | major/extensive post-processing modification. |
| 1 | Poor implant fit; optimal cosmesis cannot be achieved with post- |
| | processing modification. |

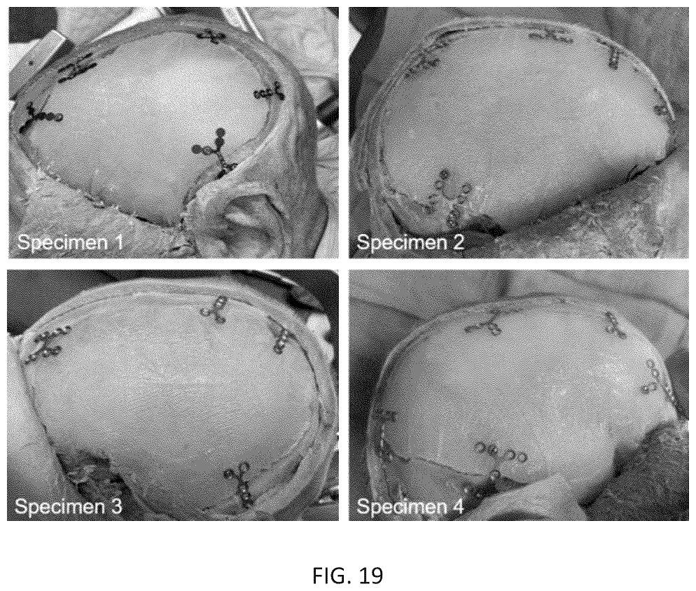

As shown in FIG. 19, in all four cadaver specimens, the molds generated from CranialRebuild produced a cranial implant with an optimal curvature using mirroring from the pristine or reference side of the skull. Modifications of the margins were required in all specimens. Minor modifications (defined as achievement of optimal cosmesis using “routine” methods of cranial implant titanium miniplate stabilization) were required in two implants, earning a score of 4/5 on the analogue scale as presented in Table 2 below. Moderate modification (defined as the need for limited drill contouring at some region of the cranial implant margin and/or use of “non-routine” mini-plate configuration) was required in the remaining two implants, which were graded at 3/5 as shown in Table 2 below. No implant required what had previously been defined as “extensive” efforts for post-processing modification. In all specimens, optimal cosmesis could be achieved. The average print time was 22 hours on the FDM printer.

TABLE 2
Visual analogue scores assigned to each cadaveric specimen.

| Specimen | VAS Evaluation of Implant Fit | VAS Score |
|---|---|---|
| 1 | Excellent implant fit, requiring minimal | 4 |
| | post-processing modification | |
| 2 | Good implant fit, requiring moderate | 3 |
| | post-processing modification | |
| 3 | Excellent implant fit, requiring minimal | 4 |
| | post-processing modification | |
| 4 | Good implant fit, requiring moderate | 3 |
| | post-processing modification | |

| | |
|---|---|
| Average Score | 3.5 |

In above Example, it was demonstrated that the fully-automated CranialRebuild program reliably facilitated production of hermetically-precise cranial implants. Optimal cosmesis could be obtained in all four specimens with only minimal or moderate post-processing modification. Minor irregularities at the margin were thought to relate, to some extent, to use of the plastic sheath to prevent methylmethacrylate adherence to the PLA two-part mold. In the Example, 3D printing of the two-part mold was utilized using PLA, a low-cost substrate. Notably, intrinsic properties of PLA predispose it to warping with autoclave sterilization. For clinical applications using a two-part mold printed in PLA, low-temperature methods of sterilization (such as with ethylene oxide or gamma irradiation, for example) would be required. Alternative substrates, such as polycarbonate, have been reported to tolerate steam autoclave without significant warping despite the higher cost of polycarbonate. Such an alternative could be considered in environments where low-temperature sterilization technologies are not available.

In view of the above, it should now be understood that at least some embodiments of the present disclosure are directed to an automatic design methodology for a patient-specific cranial implant. The methodology is based on the assumption that the skull is approximately symmetric about the midsagittal plane. It is noted that the methodology discussed herein works only when the bone flap is entirely present on either side of the midsagittal plane because of the key concept of mirroring the pristine side (reference side) to the defective side (hole side). The key advantage of the exemplary methods, systems, and programs described herein is the complete automation of the design process that makes it amenable for medical professionals who do not have any 3-D printing expertise to use the system without any special training.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method comprising:
imaging a head of a patient to generate a DICOM file including pixel data of the imaged head;
extracting a virtual skull model from the pixel data of the DICOM file, the virtual skull model having a reference side and a surgical hole side;
identifying a location of the zygomatic bone in the virtual skull model and defining an inferior boundary of the virtual cranial implant based on the location of the zygomatic bone;
determining a location of a mid-sagittal plane of the virtual skull model;
determining a distance to an inside surface and an outside surface of the virtual skull model on both the reference side and surgical hole side;
identifying a surgical hole and the surgical hole side of the virtual skull model based on the distance to the inside surface and the distance to the outside surface of the virtual skull model on both the reference side and surgical hole side of the virtual skull model;
identifying a ridge of the surgical hole on the surgical hole side of the virtual skull model;
aligning the inside surface and the outside surface of both the reference side and the surgical hole side of the virtual skull model to generate an aligned inner surface and an aligned outer surface on the reference side of the virtual skull model;
generating a virtual cranial implant based on the surgical hole, the aligned inner surface on the reference side of the virtual skull model, and the aligned outer surface on the reference side of the virtual skull model; and
generating a virtual two-part mold based on the virtual cranial implant.

2. The method of claim 1, further comprising generating an STL file from the virtual two-part mold and constructing a physical two-part mold from the STL file using an additive manufacturing apparatus.

3. The method of claim 2, further comprising constructing a physical cranial implant with the physical two-part mold.

4. The method of claim 1, wherein determining the location of the mid-sagittal plane of the virtual skull model is based on a maximum symmetry of the reference side and the surgical hole side of the virtual skull model.

5. The method of claim 1, further comprising tracing a plurality of rays from the mid-sagittal plane to both the reference side and the surgical hole side of the virtual skull model to determine the distance to the inside surface and the distance to the outside surface on both the reference side and surgical hole side.

6. The method of claim 1, further comprising using connected-component labeling to identify the surgical hole.

7. The method of claim 1, further comprising identifying a thickness of the virtual skull model based on the ridge.

8. The method of claim 1, further comprising scaling the virtual cranial implant by a user defined scale factor.

9. The method of claim 1, further comprising generating a pin-lock mechanism on the virtual two-part mold.

10. The method of claim 9, wherein the pin-lock mechanism comprises a plurality of pin-receiving members disposed on one half of the two-part mold and a plurality of pins disposed on the other half of the two-part mold.

11. The method of claim 10, further comprising controlling a thickness of a gap formed between both halves of the physical two-part mold based on the length of the plurality of pins.

12. A non-transitory computer readable medium comprising instructions that, when executed by a processor, cause the processor to perform operations comprising:
extracting pixel data from a DICOM file;
generating a virtual skull model based on the pixel data;
identifying a mid-sagittal plane of the virtual skull model;
identifying an inferior boundary of the virtual skull model;
identifying a surgical hole in the virtual skull model;
mirroring a reference side of the virtual skull model onto a surgical hole side of the virtual skull model;
subtracting the surgical hole side from the mirrored reference side to generate a virtual cranial implant; and
generating a virtual two-part mold based on the virtual cranial implant.

13. The non-transitory computer readable medium according to claim 12, wherein the operations comprise providing a graphical user interface configured to facilitate visualizing the virtual skull model, the virtual cranial implant, and the virtual two-part mold.

14. The non-transitory computer readable medium according to claim 12, wherein the operations comprise providing a graphical user interface configured to facilitate exporting the virtual skull model, the virtual cranial implant, and the virtual two-part mold.

15. The non-transitory computer readable medium according to claim 12, wherein the operations comprise providing a graphical user interface configured to facilitate scaling the virtual cranial implant by a user defined scale factor.

16. The non-transitory computer readable medium according to claim 12, wherein the operations comprise generating a pin-lock mechanism on the virtual two-part mold, the pin-lock mechanism comprising a plurality of pin-receiving members disposed on one half of the two-part mold and a plurality of pins disposed on the other half of the two-part mold.

17. The non-transitory computer readable medium according to claim 16, wherein the operations comprise providing a graphical user interface configured to facilitate specifying a length of the plurality of pins.

18. A method comprising:
receiving a DICOM file containing image data representative of an image of a patient's head through a graphical user interface coupled to a processor and displayed on a display;
generating a virtual skull model based on the image data with the processor;
identifying a mid-sagittal plane of the virtual skull model with the processor;
identifying a surgical hole in the virtual skull model with the processor;
mirroring a reference side of the virtual skull model onto a surgical hole side of the virtual skull model with the processor;
subtracting the surgical hole side from the mirrored reference side to generate a virtual cranial implant with the processor;
scaling the virtual cranial implant by a user defined scale factor through the graphical user interface;
generating a virtual two-part mold based on the scaled virtual cranial implant with the processor;
generating a pin-lock mechanism on the virtual two-part mold with the processor, the pin-lock mechanism comprising a plurality of pin-receiving members disposed on one half of the two-part mold and a plurality of pins disposed on the other half of the two-part mold;
specifying a length of the plurality of pins of the pin-lock mechanism through the graphical user interface;
generating an STL file of the virtual two-part mold and pin-lock mechanism with the processor;
printing a physical two-part mold and pin-lock mechanism from the STL file using an additive manufacturing apparatus; and
forming a physical cranial implant using the physical two-part mold and pin-lock mechanism.

19. The method of claim 18, further comprising identifying a location of the zygomatic bone in the virtual skull model with the processor and defining an inferior boundary of the virtual cranial implant based on the location of the zygomatic bone.

20. The method of claim 18, further comprising controlling a thickness of a gap formed between both halves of the physical two-part mold based on the length of the plurality of pins, wherein the thickness of the gap corresponds to a thickness of the physical cranial implant.
